(19) United States
(12) Patent Application Publication
(10) Pub. No.: US 2008/0262078 A1
(43) Pub. Date: Oct. 23, 2008

(54) Pharmaceutical Compositions

(76) Inventors: Alok B. Namdeo, Baroda (IN); N. Subramanian, Baroda (IN); Subhas Balaram Bhowmick, Baroda (IN)

Correspondence Address: PHARMACEUTICAL PATENT ATTORNEYS, LLC, 55 MADISON AVENUE, 4TH FLOOR, MORRISTOWN, NJ, 07960-7397, US

(21) Appl. No.: 12/106,355

(22) Filed: Apr. 21, 2008

(30) Foreign Application Priority Data
Apr. 20, 2007 (IN) 787/MUM/2007

(51) Int. Cl. A61K 31/337; A61P 35/00

(52) U.S. Cl. 514/449

(57) ABSTRACT

The present invention relates to a process for the preparation of a stable lyophilized form of a water insoluble drug suitable for parenteral use and pharmaceutical compositions comprising such lyophilized form of the drug.

This application claims priority from Indian Patent Application 787/MUM/2007 filed Apr. 20, 2007.

### FIELD OF THE INVENTION

The present invention relates to a process for the preparation of a stable lyophilized form of a water insoluble drug suitable for parenteral use and pharmaceutical compositions comprising such lyophilized form of the drug.

### BACKGROUND OF THE INVENTION

Parenteral preparations of water insoluble drugs are a difficult task in the pharmaceutical art. Solubilization of water insoluble drugs, using excipients that are generally regarded as safe for parenteral administration, is a complex issue, especially since conventional means of improving solubility may not always be suitable. This is more of a problem for drugs that need to be provided in the form of dosage forms for reconstitution because of stability problems associated with solution forms of the drug. Reconstitution of the drug has to be afforded in the smallest possible time and with minimal efforts, for ease of administration. The solution formed upon reconstitution should be clear, particulate-free, physically and chemically stable and should be safe and efficacious. Meeting these goals is a challenge to the formulator.

Various techniques have been used to improve solubility of water insoluble drugs. For example, the most common technique is that of size reduction of the drug to increase surface area available for interaction with the liquid medium. Size reduction may be carried out using conventional processes such as milling, grinding (with or without a liquid vehicle), precipitation into a non-solvent, and the like. However, these milled particles usually tend to agglomerate over a period of time, thereby forming aggregates that are difficult to dissolve or disperse. This problem has been taken care of by adsorbing a surface stabilizer on the surface of the comminuted drug, immediately after its size is reduced, or carrying out particle size reduction in the presence of a suitable surface stabilizer. This ensures that the particles do not agglomerate into larger aggregates. Typical examples of such techniques are disclosed in U.S. Pat. Nos. 4,107,288, 4,880,623, 5,202,129, 4,329,332, 5,560,932, 5,662,883, 5,510,118 and others. However, the use of surface stabilizer may not be suitable for parenteral preparations. Also, the process is tedious and time-consuming. Although the solubility of the drug may improve upon size reduction, the time required to solubilize or reconstitute the drug may still be an issue.

Further, it has been observed with some drugs that size reduction using conventional techniques causes the crystal lattice to fracture in a manner such that upon contact with a solvent or water during formulation of the drug, stability issues such as development of unwanted coloration, chemical degradation, may arise.

It has been a common practice in the art to form rapidly dispersible or soluble lyophilized forms of water soluble drugs by freeze drying or lyophilization, wherein the drug is dissolved in aqueous medium, the solution frozen and subjected to high vacuum whereby the ice is converted directly to vapor (sublimation) leaving a fluffy mass which is readily reconstituted. However, water insoluble drugs are not amenable to this process. The process was applied by replacing water with t-butanol in Ni et al.

Ni et al in International Journal of Pharmaceutics, 226 (2001), p 39-46 disclose intravenous injection of an antitumor drug, SarCNU, wherein the SarCNU is lyophilized in neat t-butanol to obtain a uniform cake of needle-shaped crystals. The article discloses that only 0.001% or 10 ppm of the t-butanol is left in the lyophilized cake. However, our repeated experiments of lyophilization of other drugs like docetaxel, paclitaxel and cyclosporin with t-butanol using the freeze drying cycle detailed in Ni et al, resulted in larger amounts of residual solvent, which would be harmful in a parenteral composition. Thus, the process did not work in a general manner but only worked for the drug SarCNU.

Other techniques that have been used include the addition of a polymer which is bonded to the drug. For example, United States Application published as No: 20050152979 disclose a lyophilized composition comprising a hydrophobic biologically active agent; a polymer that renders said hydrophobic active agent soluble in an aqueous solution, and a reconstitution enhancing agent, wherein time of reconstitution of said composition in an aqueous solution is less than that for said composition absent said enhancing agent. The compositions are prepared by solubilizing the hydrophobic active agent, the polymer and one or more reconstitution enhancing agents in purified water and lyophilizing the solution to obtain the finished product. This lyophilized product was found to reconstitute in less than 60 seconds in purified water, with the reconstitution enhancing agent being responsible for the fast reconstitution. However, the use of new excipients such as new polymers that have not been previously used in parenteral dosage forms, require a detailed investigation of the safety and efficacy of administering the desired quantity of the polymer directly into venous circulation by the intravenous route.

Another concern in preparing a lyophilized form of a drug is the ability of the lyophilized form to maintain its properties such as “ready solution”, low degradation impurities and absence of formation of a cake by agglomeration or crystal growth between particles.

Hence, there is need to provide an easy and simplified process for preparing a lyophilized form of a water insoluble drug which reconstitutes rapidly in a sterile liquid vehicle without the use of new additives or auxiliaries that require safety evaluation. Furthermore, the reconstituted solution on further dilution with a suitable aqueous parenteral infusion solution should not precipitate out of the solution for at least a period during which the drug is infused into body fluids. There is also a further need to provide a lyophilized form of the water insoluble drug which is stable and is substantially free of residual organic solvents so as to be suitable for parenteral administration.

### OBJECTS OF THE INVENTION

It is therefore an object of the present invention to provide a process for the preparation of a stable lyophilized form of a water insoluble drug suitable for parenteral use and pharmaceutical compositions comprising such lyophilized form of the drug.

It is another object of the present invention to provide a stable lyophilized form of a water insoluble drug that is reconstituted into a solution in a suitable parenterally acceptable vehicle conveniently and rapidly.

It is another object of the invention to provide a stable lyophilized form of water insoluble drug which after reconstitution in a parenterally acceptable vehicle to form a solution, is further diluted with an aqueous infusion vehicle without precipitation.

It is still another object of the present invention to provide a stable lyophilized docetaxel.

It is yet another object of the present invention to provide a sterile composition comprising a stable lyophilized form of a water insoluble drug.

It is further object of the present invention to provide a sterile composition prepared by adding a sterile liquid vehicle consisting essentially of a solubilizer and a solvent selected from organic compounds having a hydroxyl group and molecular weight less than 200, to the stable lyophilized form of a water insoluble drug.

It is yet another object of the present invention to provide a kit comprising a sterile composition comprising the stable lyophilized form of a water insoluble drug, prepared by the process of the present invention in a first container, and a sterile liquid vehicle consisting essentially of a solubilizer and a solvent in a second container.

It is yet another object of the present invention to provide an infusion solution prepared by a process comprising diluting the composition in the kit comprising the sterile composition of a stable lyophilized water insoluble drug in a liquid vehicle consisting essentially of a solubilizer and a solvent selected from organic compounds having a hydroxyl group and molecular weight less than 200, with an aqueous infusion vehicle.

Still other objects of the invention will be apparent to those of ordinary skilled in the art.

### SUMMARY OF THE INVENTION

In one aspect of the invention, there is provided a process for the preparation of a stable lyophilized form of a water insoluble drug suitable for parenteral use, said process comprising:
a) mixing the water insoluble drug with a sufficient quantity of ethanol to dissolve said drug,
b) sterilizing the solution
c) precipitating the drug by adding sufficient quantity of sterile water, and
d) subjecting the sterile suspension so obtained to lyophilization.

In another aspect of the invention there is provided a stable lyophilized form of a water insoluble drug that is reconstituted into a solution in a suitable parenterally acceptable vehicle conveniently and rapidly.

In another aspect of the invention there is provided a stable lyophilized form of water insoluble drug which after reconstitution in a parenterally acceptable vehicle to form a solution, is further diluted with an aqueous infusion vehicle without precipitation.

In yet another aspect of the invention there is provided a stable lyophilized docetaxel.

In another aspect of the invention there is provided a sterile composition comprising a stable lyophilized form of a water insoluble drug.

In a still further aspect of the invention there is provided a sterile composition prepared by adding a sterile liquid vehicle consisting essentially of a solubilizer and a solvent selected from organic compounds having a hydroxyl group and molecular weight less than 200, to a stable lyophilized form of a water insoluble drug.

In another aspect of the invention there is provided a kit comprising a sterile composition comprising the stable lyophilized form of a water insoluble drug, prepared by the process of the present invention in a first container, and a sterile liquid vehicle consisting essentially of a solubilizer and a solvent in a second container.

In another aspect of the invention there is provided an infusion solution prepared by a process comprising diluting the composition in the kit comprising the sterile composition of stable lyophilized water insoluble drug in a liquid vehicle consisting essentially of a solubilizer and a solvent selected from organic compounds having a hydroxyl group and molecular weight less than 200, with an aqueous infusion vehicle.

### BRIEF DESCRIPTION OF THE DRAWINGS

Many aspects of the invention can be better understood with reference to the following drawings. The components in the drawings are not necessarily to scale, emphasis instead being placed upon clearly illustrating the principles of the present invention.

FIG. 1: XRD of the lyophilized form of docetaxel obtained in Example 2, after lyophilization.

FIG. 2: XRD of the lyophilized form of docetaxel obtained in Example 2, after storage at 25±2° C., 60±5% RH and analyzed at 3 months.

FIG. 3: XRD of the lyophilized form of docetaxel obtained in Example 2, after storage at 25±2° C., 60±5% RH and analyzed at 6 months.

### DETAILED DESCRIPTION OF THE INVENTION

The present invention provides a process for the preparation of a stable lyophilized form of a water insoluble drug suitable for parenteral use, said process comprising
a) mixing the water insoluble drug with a sufficient quantity of ethanol to dissolve said drug,
b) sterilizing the solution
c) precipitating the drug by adding sufficient quantity of sterile water, and
d) subjecting the sterile suspension so obtained to lyophilization.

The term “water insoluble drug” as used herein includes drugs that dissolve with difficulty, i.e., the water insoluble drug that requires more than 120 seconds to form a clear solution (i.e. presence of no visible particles) when 20 mg of the water insoluble drug is mixed with a sterile liquid vehicle consisting essentially of 520 mg of Polysorbate 80 and 0.2 ml of ethanol, such as, for example, when mixed in a vial and the vial agitated manually, or when mixed in a vial and the vial agitated in a rotating bottle apparatus at 50 rpm, or when mixed in a vial and the vial agitated in a multipulse shaker at 50 rpm. Examples of such drugs include, but are not limited to taxoids such as docetaxel and paclitaxel, steroids such as flunisolide, cyclosporine, and their pharmaceutically acceptable salts, derivatives, analogs and isomers.

The term “lyophilized form” as used herein refers to a form of the water insoluble drug that is free of any added excipients, is reconstituted into a solution readily in a sterile liquid vehicle suitable for parenteral administration. The ready reconstitution into a solution may be tested using the same test as described above except that the “lyophilized form” requires less than 120 seconds to form a clear solution.

The term “stable” as used herein refers to the lyophilized form of the water insoluble drug which when packed in vials and stored at 25±2° C., 60±5% relative humidity for a period of 6 months, the amount of total impurities are less than 3.0%. Further, the term “stable” as used herein also refers to the lyophilized form of the water insoluble drug which when reconstituted in a sterile liquid vehicle, reconstitutes in less than 120 seconds and the reconstituted solutions are clear without precipitation of the water insoluble drug for at least 2 hours after addition of the sterile liquid vehicle.

The term “suitable for parenteral use” as used herein refers to the lyophilized form water insoluble drug which is substantially free of residual organic solvents and other impurities and which is safe for human administration through an injectable route.

A preferred drug for the present invention is docetaxel. Docetaxel is an antineoplastic agent belonging to the taxoid family. It is prepared by semisynthesis beginning with a precursor extracted from the renewable needle biomass of yew plants. The chemical name for docetaxel is (2R,3S)-N-carboxy-3-phenylisoserine,N-tert-butyl ester, 13-ester with 5β-20-epoxy-1,2α,4,7β,10β,13α-hexahydroxytax-11-en-9-one 4-acetate 2-benzoate, Docetaxel is marketed in the United States of America as TAXOTERE® injection concentrate. TAXOTERE (docetaxel) Injection Concentrate is a clear yellow to brownish-yellow viscous solution. TAXOTERE is sterile, non-pyrogenic, and is available in single-dose vials containing 20 mg (0.5 mL) or 80 mg (2 mL) docetaxel (anhydrous). Each mL contains 40 mg docetaxel (anhydrous) and 1040 mg polysorbate 80. TAXOTERE Injection Concentrate requires dilution prior to use. TAXOTERE as a single agent is indicated for the treatment of patients with locally advanced or metastatic non-small cell lung cancer after failure of prior platinum-based chemotherapy. TAXOTERE in combination with cisplatin is indicated for the treatment of patients with unresectable, locally advanced or metastatic non-small cell lung cancer who have not previously received chemotherapy for this condition. TAXOTERE in combination with prednisone is indicated for the treatment of patients with androgen independent (hormone refractory) metastatic prostate cancer. TAXOTERE in combination with cisplatin and fluorouracil is indicated for the treatment of patients with advanced gastric adenocarcinoma, including adenocarcinoma of the gastroesophageal junction, who have not received prior chemotherapy for advanced disease. TAXOTERE in combination with cisplatin and fluorouracil is indicated for the induction treatment of patients with inoperable locally advanced squamous cell carcinoma of the head and neck Paclitaxel is a natural product with antitumor activity. Paclitaxel is obtained via a semisynthetic process from Taxus baccata. The chemical name for paclitaxel is 5beta,20-epoxy-1,2alpha,4,7beta,10beta,13alpha-hexahydroxytax-11-en-9-one 4,10-diacetate 2-benzoate 13-ester with (2R,3S )-N-benzoyl-3-phenylisoserine. It is marketed in the United Sates of America as TAXOL Injection. TAXOL is supplied as a nonaqueous solution intended for dilution with a suitable parenteral fluid prior to intravenous infusion. TAXOL is indicated as first-line and subsequent therapy for the treatment of advanced carcinoma of the ovary. As first-line therapy, TAXOL is indicated in combination with cisplatin. TAXOL is indicated for the adjuvant treatment of node-positive breast cancer administered sequentially to standard doxorubicin-containing combination chemotherapy. TAXOL is indicated for the treatment of breast cancer after failure of combination chemotherapy for metastatic disease or relapse within 6 months of adjuvant chemotherapy. TAXOL, in combination with cisplatin, is indicated for the first-line treatment of non-small cell lung cancer in patients who are not candidates for potentially curative surgery and/or radiation therapy. TAXOL is indicated for the second-line treatment of AIDS-related Kaposi's sarcoma.

Cyclosporine is a cyclic polypeptide immunosuppressant agent consisting of 11 amino acids. It is produced as a metabolite by the fungus species Beauveria nivea. Chemically, cyclosporine is designated as [R—[R*,R*-(E)]]-cyclic(L-alanyl-D-alanyl-N-methyl-L-leucyl-N-methyl-L-leucyl-N-methyl-L-valyl-3-hydroxy-N,4-dimethyl-L-2-amino-6-octenoyl-L-α-amino-butyryl-N methylglycyl-N-methyl-L-leucyl-L-valyl-N-methyl-Lleucyl). It is available in the United States of America as Sandimmune® Injection. Sandimmune® Injection is available in a 5 mL sterile ampul for I.V. administration. Sandimmune® (cyclosporine) is indicated for the prophylaxis of organ rejection in kidney, liver, and heart allogeneic transplants.

The present invention provides a process for the preparation of a stable lyophilized form of a water insoluble drug suitable for parenteral use in order to provide means for improving solubility of water insoluble drugs by converting the drug to a physical form that is suitable for dissolving and readily forming a clear solution in the desired liquid vehicle suitable for parenteral administration. This physical form of the drug (also referred to herein as lyophilized form of the drug) is capable of dissolving in the liquid vehicle in less than 120 seconds with minimal efforts. For example, upon conversion to the suitable physical form, the drug dissolves in the liquid vehicle immediately upon addition of the liquid vehicle, or with minimum agitation of the container by the professional personnel reconstituting the drug composition, with a sterile liquid vehicle suitable for parenteral administration, and/or with an aqueous infusion vehicle. Further, the use of the suitable physical form of the drug ensures that the drug stays in solution in the liquid vehicle for at least 2 hours after reconstitution. Preferably, the drug stays in solution for about 8 hours after reconstitution, when stored under normal conditions of storage, such as ambient room temperature.

The term “sterile liquid vehicle suitable for parenteral administration” (this term has been used interchangeably with “sterile liquid vehicle” and “liquid vehicle”) as used herein means a vehicle that is capable of dissolving the stable lyophilized form of the water insoluble drug, and which is suitable for parenteral administration, without causing any adverse events to the patient. The sterile liquid vehicle consists essentially of a solubilizer and a solvent selected from organic compounds having a hydroxyl group and molecular weight less than 200.

Examples of solvent suitable for use in the sterile liquid vehicle of the present invention include, but are not limited to, alcohols like ethanol, benzyl alcohol, isopropyl alcohol, and the like, propylene glycol, polyethylene glycol, and the like and mixtures thereof. The solubilizer suitable for use in the sterile liquid vehicle include, but are not limited, to polyoxyethylene sorbitan fatty acid esters, polyoxyethylene castor oil derivatives, fatty acid-polyethylene glycol esters, vitamin E tocopherol propylene glycol succinate (Vitamin E TPGS), sucrose-fatty acid esters and the like and mixtures thereof. The solvents and solubilizers used are those that have been used in marketed preparations administered to human subjects.

Polyoxyethylene sorbitan fatty acid esters that can be used as solubilizer in the liquid vehicle of the present invention may be selected from polyoxyethylene 20 sorbitan monolaurate (Polysorbate 20), polyoxyethylene 20 sorbitan monopalmitate (Polysorbate 40), polyoxyethylene 20 sorbitan monooleate (Polysorbate 80) and mixtures thereof. These polyoxyethylene sorbitan fatty acid esters (polysorbates) are a series of partial fatty acid esters of sorbitol and its anhydrides copolymerized with approximately 20 moles of ethylene oxide for each mole of sorbitol and its anhydrides. Preferably, the polysorbate of choice is polysorbate 80 having a saponification value in the range of 45-55, moisture content of 3% or less, hydroxyl value of 65-80 and an acid value of 2% or less. It may be used in an amount ranging from about 250 mg per ml of the liquid vehicle to about 1000 mg per ml of the liquid vehicle.

Polyoxyethylene castor oil derivatives are a series of materials obtained by reacting varying amounts of ethylene oxide with either castor oil or hydrogenated castor oil, thereby forming a complex mixture of hydrophobic and hydrophilic components.

They mainly contain ricinoleyl glycerol ethoxylated with 30-50 molecules of ethylene oxide. Commercially available grade of polyoxyl 40 hydrogenated castor oil, Cremophor RH 40, is preferred as the liquid vehicle, having a moisture content of 2% or less, saponification value of 45-69, iodine value of 2.0 or less and a hydroxyl value of 60-80.

The sterile liquid vehicle is used in an amount sufficient to dissolve the stable lyophilized form of the water insoluble drug suitable for parenteral use, and in an amount that is safe and non-toxic for parenteral administration. Preferably, the liquid vehicle used, and the amount in which it is used, is selected such that a stable composition is obtained, i.e. a composition that does not precipitate the drug for at least 2 hours after the liquid vehicle has been added to the drug. In one embodiment of the present invention, 520 mg of polysorbate 80 is used in combination with 0.2 ml of ethanol as the liquid vehicle. The sterile liquid vehicle of the present invention may be provided in a separate container. The vehicle may be filled into unit dose containers and subjected to sterilization. Sterilization may be carried out in any of the conventional methods known in the art, such as, steam sterilization, dry heat sterilization, radiation sterilization, sterile filtration, or any other means of sterilization that is suitable for the particular liquid vehicle being used.

In the process of the invention, the water insoluble drug is converted to a physical form suitable for dissolving and readily forming a clear solution in the sterile liquid vehicle, by first mixing the water insoluble drug with a sufficient quantity of ethanol to dissolve the drug and then sterilizing the resultant solution. Sterilization may be typically done by membrane filtration of the solution. Alternatively, it may be sterilized by any other conventional means of sterilization as may be suitable for the sterile liquid vehicle. A sufficient quantity of sterile water, or other sterile non-solvent, is then added to precipitate the drug out of the solution. The sterile suspension so obtained is subjected to lyophilization. The lyophilized product obtained is suitable for dissolving and readily forming a clear solution in the sterile liquid vehicle.

In an embodiment, docetaxel is dissolved in ethanol in amount such that the concentration of docetaxel in ethanol ranges from about 90 mg/ml to about 98 mg/ml. Docetaxel is precipitated out of the ethanol by adding sterile water at room temperature. The ratio of ethanol to water ranges from about 1:1 to about 1:10, preferably the ratio is about 1:5.

Lyophilization or freeze-drying may be performed using commercial freeze-dryers, such as are available from a variety of sources, using manufacturer recommended settings. Typically, the product is freeze-dried so that the stable lyophilized product contains less than about 3000 ppm of the organic solvent. In processes where an aqueous suspension is subjected to lyophilization, the product is freeze-dried so that less than about 1.5% w/v moisture is present. In one example, the product is loaded at about 5° C., frozen to about −40° C. and held at −40° C. for about seven to about eight hours; the frozen solution is then thermally treated by raising the shelf temperature to −20° C. to −25° C., and holding at that temperature for 2 to 8 hours. Thereafter, the condenser can be started, the vacuum adjusted and the shelf temperature raised to +25° C. Optionally, when the product temperature reaches +25° C., the product is subjected to secondary drying. Suitably, the lyophilization process results in a product having residual solvent in an amount of less than 2% by weight of the final weight of solids in the lyophilized product. In addition or alternatively to the second step, other processing techniques can be used to further reduce the residual solvent in the resulting lyophilized material. Such processing techniques include nitrogen sweeps, among other methods.

The lyophilization may be carried out in bulk or in unit dose containers. For example, the sterile suspension of the water insoluble drug obtained upon addition of sterile water may be subjected to bulk lyophilization, followed by aseptic filling of the required amount of the lyophilized product into sterile unit dose containers. This is typically referred to as dry powder filling. The cake obtained on bulk lyophilization may be subjected to mechanical sieving under aseptic conditions, prior to filling into unit dose containers, so as to break any agglomerates and facilitate easy filling of the required amount of the product into sterile containers. Alternatively, the suspension of the water insoluble drug may be filled into unit dose containers, with each container containing an equal amount of the suspension. These individual containers may then be subjected to lyophilization, so that the lyophilized product is obtained in the unit dose containers. In one embodiment of the present invention, the sterile solution of the water insoluble drug in ethanol is filled aseptically into sterile unit dose containers and the required quantity of sterile water is added to each container to precipitate the drug, i.e. to form a suspension. The unit dose containers containing the sterile suspension are then lyophilized. In the process of the invention, the lyophilization is carried out by subjecting the sterile suspension of the water-insoluble drug to lyophilization, such that the lyophilized form contains less than about 3000 ppm of the residual organic solvent. In a preferred embodiment of the invention, the lyophilization is carried out by subjecting the sterile suspension of the water-insoluble drug to lyophilization, such that the lyophilized form contains less than about 3000 ppm of ethanol.

Stable lyophilized form of the water insoluble drug prepared by the process of the present invention was packed in vials and stored at 25±2° C., 60±5% relative humidity for period of six months. The vials were analyzed using High Performance Liquid Chromatography (HPLC) for the amount of total impurities and the assay at an interval of 1, 2, 3 and 6 months. The lyophilized water insoluble drug was found to have less than 3.0% total impurities, after storage for 6 months at 25±2° C., 60±5% relative humidity.

In one embodiment of the invention, there is provided a stable lyophilized docetaxel. The lyophilized docetaxel has particularly good pharmaceutical properties. It is particularly stable and has a moisture content of not more than 3.0%. The lyophilized docetaxel has good storage properties and can be rapidly reconstituted with a sterile liquid vehicle without the use of new additives or auxiliaries that require safety evaluation. Further more, the reconstituted solution on further dilution with a suitable aqueous parenteral infusion solution does not precipitate out of the solution for at least a period during which the drug is infused into body fluids. The stable lyophilized docetaxel has a residual organic solvent less than 3000 ppm. Preferably, the organic solvent is ethanol.

In one embodiment of the present invention, a kit is provided which comprises:
a sterile composition comprising the stable lyophilized form of a water insoluble drug suitable for parenteral use prepared by the process as described herein in a first container, and
a sterile liquid vehicle consisting essentially of a solubilizer and a solvent selected from organic compounds having a hydroxyl group and molecular weight less than 200, in a second container.

The sterile composition comprising the stable lyophilized form of a water insoluble drug suitable for parenteral use in a first container is dissolved readily upon addition of the sterile liquid vehicle provided in the second container. Typically, the stable lyophilized form of the water insoluble drug suitable for parenteral use obtained by the process of the present invention dissolves in less than 180 seconds, upon addition of the sterile liquid vehicle. Preferably, the stable lyophilized form of the water insoluble drug dissolves in less than 120 seconds.

In one embodiment of the present invention, an infusion solution is provided by diluting the sterile composition i.e. the stable lyophilized form of a water insoluble drug suitable for parenteral use, dissolved in the sterile liquid vehicle, with an aqueous infusion vehicle. Examples of such aqueous infusion vehicles include, but are not limited to, 5% dextrose solution, 0.9% physiological saline, sterile water for injection, and the like, conventionally used in hospitals for administration. The choice of the infusion that may be used for diluting the sterile composition of the invention depends on the compatibility of the drug with the infusion solution to be used.

While some embodiments seek to avoid use of auxillary excipients in the process of lyophilization of the present invention, not all auxillary excipients need to be eliminated. Thus as desired, the solution prepared prior to any precipitation of the material and prior to any freeze drying step can have suitable auxiliary excipients like for example, antioxidants, chelating agents, tonicity agents, buffers, pH-adjusting agents, cryoprotectants, bulking agents that aid in lyophilization, diluents and various other pharmaceutical agents conventionally used in parenteral formulations may be used in amounts conventional to the pharmaceutical art.

In a preferred embodiment of the present invention, a taxane derivative is used as the water insoluble drug to obtain the pharmaceutical composition of the present invention. Particularly, docetaxel is used as the preferred taxane derivative to provide the stable lyophilized docetaxel. Prior art parenteral formulations of docetaxel were obtained by dissolving docetaxel in a mixture of Cremophor and ethanol (as disclosed in example 1 of U.S. Pat. No. 4,814,470). However, the solubility of docetaxel is so poor, that a large amount of Cremophor and ethanol, i.e. 50% by volume of Cremophor and 50% by volume of ethanol, is required to obtain a solution formulation. The high quantities of Cremophor were found to cause anaphylactic reactions in patients, while the high amount of ethanol caused alcoholism (see Rowinsky, Lorraine, Cazenave and Donehower, Journal of the National Cancer Institute, vol. 82, No. 15, pages 1247 to 1259). In order to avoid these problems associated with Cremophor and ethanol, a new formulation was prepared by Rhone-Poulenc Rorer (as described in U.S. Pat. No. 5,438,072), which was essentially free of ethanol, and which replaced Cremophor with another surfactant selected from polysorbates, ethylene oxide esters-ethers and fatty acids glycerides. The commercial product, Taxotere®, includes Polysorbate 80 as the surfactant. The product is available in the form of a kit—a first vial containing a solution of docetaxel in Polysorbate 80, and a second vial containing water with 13% ethanol, as the diluent. The present invention provides, in a preferred embodiment, a stable pharmaceutical composition of docetaxel (see examples 1 and 2 below), which is easy to reconstitute prior to administration, and which overcomes the disadvantages of the prior art, such as alcoholism and anaphylactic reactions.

The examples that follow do not limit the scope of the present invention and are merely used as illustrations.

### COMPARATIVE EXAMPLE 1

Docetaxel (20 mg) was dissolved in tertiary Butanol (1 ml) at a controlled temperature of 25±2° C. The solution thus obtained was then sterile filtered, filled in sterile vials and lyophilized to form a white cake. The lyophilization cycle detailed in Table 1 below was used.

TABLE 1

| Temp | Time | | | Pressure | | Time | | | | Total time | | |
|---|---|---|---|---|---|---|---|---|---|---|---|---|
| □C. | HH | MM | SS | No. | % | HH | MM | SS | Stage | HH | MM | SS |
| +5 | 01 | 00 | 00 | | | | | | Freezing | 07 | 30 | 00 |
| −40 | 01 | 30 | 00 | | | | | | | | | |
| −40 | 05 | 00 | 00 | | | | | | cycle | | | |
| −40 | 01 | 00 | 00 | | | | | | Preparation | 01 | 00 | 00 |
| | | | | | | | | | for | | | |
| | | | | | | | | | drying | | | |
| −40 | 00 | 10 | 00 | 1 | 40 | 00 | 10 | 00 | Primary | 42 | 50 | 00 |
| −20 | 00 | 40 | 00 | 2 | 36 | 10 | 40 | 00 | Drying | | | |
| −20 | 10 | 00 | 00 | 3 | 30 | 32 | 00 | 00 | | | | |
| 0 | 03 | 20 | 00 | | | | | | | | | |
| 0 | 05 | 00 | 00 | | | | | | | | | |
| 25 | 04 | 10 | 00 | | | | | | | | | |
| 25 | 20 | 00 | 00 | | | | | | | | | |
| 25 | 00 | 00 | 01 | | | | | | Secondary | 00 | 00 | 01 |
| | | | | | | | | | drying | | | |

Extend Secondary drying at +25° C. and 30% pressure, until moisture content is achieved at below 1% & t-butanol content below 50000 ppm.

The lyophilized docetaxel thus obtained had a residual solvent (t-butanol) content of about 50000 ppm and it was difficult to reduce this to less than 5000 ppm. Such high amounts of residual solvent would obviously be unacceptable for use.

### EXAMPLE 1

A pharmaceutical composition according to the present invention was prepared as mentioned below to provide a final dosage form comprising 20 mg of lyophilized docetaxel per vial.

Docetaxel (97.6 mg) was dissolved in 1 ml of ethanol, i.e. dehydrated alcohol with stirring at medium speed. The solution thus obtained was sterile filtered through membrane filter, and 0.25 ml of the filtered solution was filled into a sterile vial. To this was added 1.25 ml of water for injection, and the suspension thus obtained was lyophilized by the lyophilization cycle detailed in Table 2 below. Lyophilization was carried out till water content was below 1.55%, and ethanol content was below 3000 ppm. A porous cake was obtained upon lyophilization.

TABLE 2

| Temp | Time | | | Pressure | | Time | | | | Total time | | |
|---|---|---|---|---|---|---|---|---|---|---|---|---|
| ° C. | HH | MM | SS | No. | μ bar | HH | MM | SS | Stage | HH | MM | SS |
| +5 | 00 | 30 | 00 | | | | | | Freezing | 06 | 00 | 00 |
| −40 | 01 | 30 | 00 | | | | | | cycle | | | |
| −40 | 04 | 00 | 00 | | | | | | | | | |
| −40 | 01 | 00 | 00 | | | | | | Preparation | 01 | 00 | 00 |
| | | | | | | | | | for | | | |
| | | | | | | | | | drying | | | |
| −40 | 30 | 00 | 00 | 1 | 933 | 10 | 00 | 00 | Primary | 123 | 50 | 00 |
| −35 | 00 | 50 | 00 | 2 | 700 | 10 | 00 | 00 | Drying | | | |
| −35 | 10 | 00 | 00 | 3 | 550 | 12 | 30 | 00 | | | | |
| −30 | 00 | 50 | 00 | 4 | 420 | 12 | 10 | 00 | | | | |
| −30 | 23 | 00 | 00 | 5 | 300 | 10 | 00 | 00 | | | | |
| −25 | 00 | 50 | 00 | 6 | 266 | 25 | 00 | 00 | | | | |
| −25 | 10 | 00 | 00 | 7 | 200 | 08 | 20 | 00 | | | | |
| 00 | 04 | 10 | 00 | 8 | 133 | 05 | 00 | 00 | | | | |
| 00 | 03 | 20 | 00 | 9 | 66 | 30 | 00 | 00 | | | | |
| 30 | 05 | 00 | 00 | | | | | | | | | |
| 30 | 35 | 00 | 00 | | | | | | Secondary | 00 | 00 | 01 |
| | | | | | | | | | drying | | | |

Secondary drying extended until water content is achieved at below 1.5% & ethanol content below 3000 ppm

### EXAMPLE 2

A pharmaceutical composition according to the present invention was prepared as mentioned below to provide a final dosage form comprising 80 mg of lyophilized docetaxel per vial.

Docetaxel (94.4 mg) was dissolved in 1 ml of ethanol, i.e. dehydrated alcohol with stirring at medium speed. The solution thus obtained was sterile filtered through membrane filter, and 1.0 ml of the filtered solution was filled into a sterile vial. To this was added 5.0 ml of water for injection, and the suspension thus obtained was lyophilized by the lyophilization cycle detailed in Table 3 below. Lyophilization was carried out till water content was below 1.55%, and ethanol content was below 3000 ppm. A porous cake was obtained upon lyophilization.

TABLE 3

| Temp | Time | | | Pressure | | Time | | | | Total time | | |
|---|---|---|---|---|---|---|---|---|---|---|---|---|
| ° C. | HH | MM | SS | No. | μ bar | HH | MM | SS | Stage | HH | MM | SS |
| +5 | 00 | 30 | 00 | | | | | | Freezing | 06 | 00 | 00 |
| −40 | 01 | 30 | 00 | | | | | | cycle | | | |
| −40 | 04 | 00 | 00 | | | | | | | | | |
| −40 | 01 | 00 | 00 | | | | | | Preparation | 01 | 00 | 00 |
| | | | | | | | | | for | | | |
| | | | | | | | | | drying | | | |
| −40 | 30 | 00 | 00 | 1 | 933 | 10 | 00 | 00 | Primary | 123 | 50 | 00 |
| −35 | 00 | 50 | 00 | 2 | 700 | 10 | 00 | 00 | Drying | | | |
| −35 | 10 | 00 | 00 | 3 | 550 | 12 | 30 | 00 | | | | |
| −30 | 00 | 50 | 00 | 4 | 420 | 12 | 10 | 00 | | | | |
| −30 | 23 | 00 | 00 | 5 | 300 | 10 | 00 | 00 | | | | |
| −25 | 00 | 50 | 00 | 6 | 266 | 25 | 00 | 00 | | | | |
| −25 | 10 | 00 | 00 | 7 | 200 | 08 | 20 | 00 | | | | |
| 00 | 04 | 10 | 00 | 8 | 133 | 05 | 00 | 00 | | | | |
| 00 | 03 | 20 | 00 | 9 | 66 | 30 | 00 | 00 | | | | |
| 30 | 05 | 00 | 00 | | | | | | | | | |
| 30 | 35 | 00 | 00 | | | | | | Secondary | 00 | 00 | 01 |
| | | | | | | | | | drying | | | |

Secondary drying extended until water content is achieved at below 1.5% & ethanol content below 3000 ppm.

The composition was provided in the form of a kit comprising a first vial containing 80 mg docetaxel, as obtained by the lyophilization cycle described above, and a second vial containing a mixture of 64.6% w/w Polysorbate 80 and 35.4% w/w of ethanol. The porous docetaxel cake of the first vial is dissolved in the mixture of polysorbate 80 and ethanol (of the second vial) in less than 90 seconds to obtain a clear solution that can be used as the stock solution to prepare further dilutions, as the need may be.

### EXAMPLE 3

A comparative solubility study was performed using lyophilized form of docetaxel, obtained as in Example 1 above and a non-lyophilized form of docetaxel, to check the time taken for complete solubilization in liquid vehicle consisting of polysorbate 80 and ethanol. The experiment was carried out at controlled room temperature (25±2° C.) using bottle rotating apparatus and multipulse shaker. The time taken for complete solubilization (i.e., formation of a clear solution with presence of no visible particles) of docetaxel was noted and is given in Table 4 below.

Experimental Details:
(a) Bottle Rotating Apparatus: 20 mg of Docetaxel was taken in a 5 ml vial. To this, 0.7 ml of liquid vehicle was added. The vial was then kept in the bottle rotating apparatus and rotated at 50 RPM. Continuous monitoring was done to check the solubilization of the drug.
(b) Multipulse Shaker Apparatus: 20 mg of Docetaxel was taken in a test tube with stopper. To this, 0.7 ml of liquid vehicle was added. The test tube was then kept in a multipulse shaker and rotated at 50 RPM. Continuous monitoring was done to check the solubilization of the drug.

TABLE 4

Time taken for complete
solubilization

| | Bottle Rotating | Multipulse |
|---|---|---|
| Description | Apparatus | Shaker |
| Docetaxel (non- | 10.67 ± 1.53 min | 9 ± 2 min |
| lyophilized) | | |
| Docetaxel | Less than 120 sec | Less than 120 sec |
| (Lyophilized, as in | | |
| Example 1) | | |

It can be observed from the above study that the time taken for complete solubilization of lyophilized form of Docetaxel is significantly lesser than the non-lyophilized form of Docetaxel.

### EXAMPLE 4

Pharmaceutical compositions according to the present invention containing 80 mg/vial of docetaxel prepared as in Example 2 above, and were packed in vials and stored at 25±2° C., 60±5% relative humidity (% RH) for a period up to six months. The samples were analyzed using high performance liquid chromatography (HPLC). The samples were also analyzed for the time taken for complete solubilization (reconstitution time) when reconstituted with a sterile liquid vehicle of polysorbate 80 and ethanol. The parameters used in the analysis are given below. The percent total impurities,assay and the reconstitution time results are summarized in Table 5 below.

### For Assay/Content of Docetaxel:

Column: YMC-Pack ODS-AQ (150 mm×4.6 mm), 3μ (YMC Corporation, JAPAN)
Flow rate: 1.0 ml/min
Column temperature: 40° C.
Detection: UV at 230 nm
Injection volume: 20 μl
Retention time: about 17 minutes
Run time: about 35 min
Mobile Phase: Water and acetonitrile mixed in the ratio of 550 ml:450 ml.
Diluent: Water and acetonitrile mixed in the ratio of 1:1
Standard preparation: 12.5 mg (11.25-13.75 mg) of docetaxel mixed with the diluent to 25 ml, sonicated, and 5 ml of this solution further diluted to 50 ml with diluent.
Test preparation: Five vials of docetaxel for injection constituted with 10 ml of diluent separately, and the contents of all these constituted vials mixed and constituted to 50 ml with the diluent. 5 ml of this constituted solution further diluted to 200 ml with diluent.

### For Related Substances:

Column: Waters Sunfire C18, 150 mm×4.6 mm, 3.5μ (Waters Corporation, USA)
Flow rate: 1.2 ml/minute
Column temperature: 40° C.
Detection: UV at 230 nm
Injection volume: 10 μl
Run time: 53 minutes
Mobile phase A: Water, filtered through 0.45μ filter paper.
Mobile phase B: Acetonitrile, filtered through 0.45μ filter paper.
Retention time: about 19 minutes
Standard preparation: 5 mg of docetaxel mixed with the diluent to 100 ml, and 2 ml of this solution further diluted to 100 ml with diluent.
Test preparation: Five vials of docetaxel for injection constituted with 5 ml of diluent separately, and the contents of all these constituted vials mixed and constituted to 100 ml with the diluent.

TABLE 5

| | | | Total | |
|---|---|---|---|---|
| Storage | Time | Assay of | impurities | Reconstitution |
| Condition | period | docetaxel (%) | (%) | time (Sec) |
| 25 ± 2° C., | Initial | 98.5 | 0.246 | 90 |
| 60 ± 5 | 1 month | 95.2 | 0.237 | 90 |
| % RH | 2 month | 93.9 | 0.350 | 90 |
| | 3 month | 95.1 | 0.234 | 85 |
| | 6 month | 96.7 | 0.261 | 82 |

It can be observed from the above study that the lyophilized docetaxel is stable after storage at 25±2° C., 60±5% relative humidity for a period of 6 months and also the lyophilized form of docetaxel reconstituted rapidly (i.e. in less than 120 seconds) after reconstituting with a sterile liquid vehicle.

### EXAMPLE 5

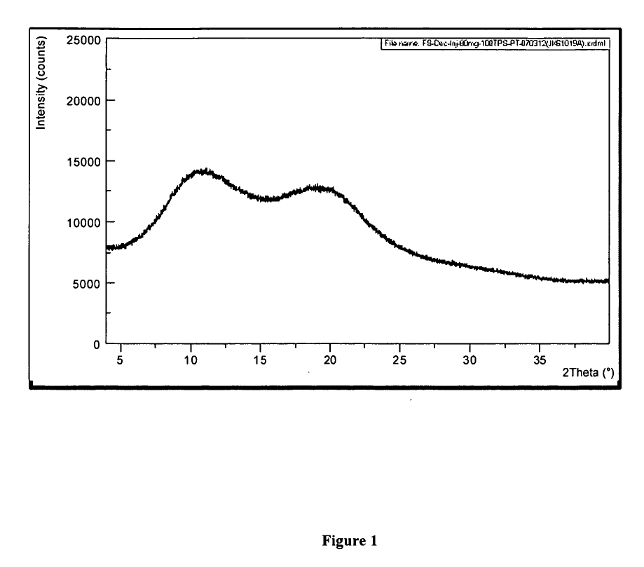

The lyophilized form of docetaxel obtained in Example 2 was analyzed by XRD. FIG. 1 shows the XRD after lyophilization.

### EXAMPLE 6

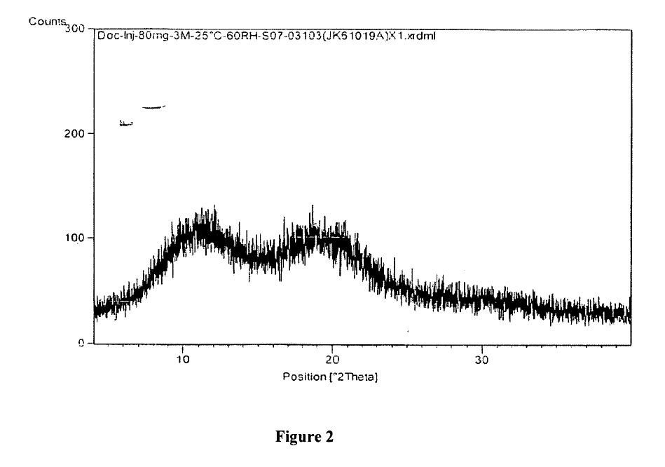

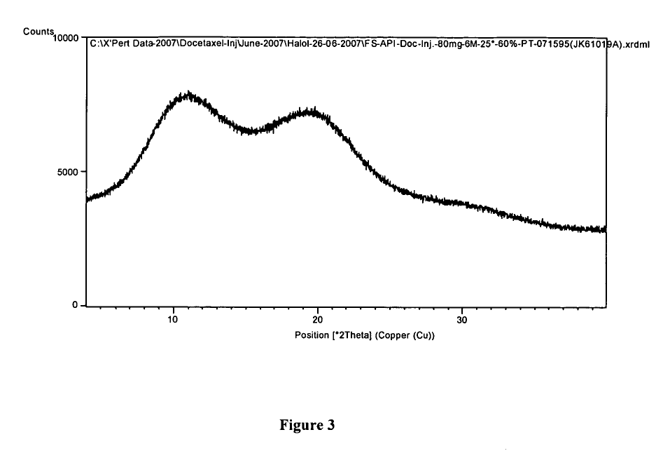

The lyophilized form of docetaxel obtained in Example 2 was analyzed by XRD. FIG. 2 and FIG. 3 shows the XRD of the lyophilized form of docetaxel after storage at 25±2° C., 60±5% RH and analyzed at 3 and 6 months respectively.

While the invention has been described by reference to specific embodiments, this was done for purposes of illustration only and should not be construed to limit the spirit or the scope of the invention.

We claim:

1. A process for the preparation of a stable lyophilized form of a water insoluble drug suitable for parenteral use, said process comprising:
a. mixing the water insoluble drug with a sufficient quantity of ethanol to dissolve said drug;
b. sterilizing the solution;
c. precipitating the drug by adding sufficient quantity of sterile water; and
d. subjecting the sterile suspension so obtained to lyophilization.

2. A process as in claim 1, wherein the water insoluble drug is selected from the group consisting of docetaxel, paclitaxel, cyclosporine and flunisolide.

3. A process as in claim 1, wherein the suspension to be lyophilized is filled into unit dose containers and lyophilized.

4. A process as in claim 1, wherein the lyophilized form of the water insoluble drug is dry powder filled into unit dose containers.

5. A process as in claim 1, wherein the drug is docetaxel, the concentration of docetaxel in ethanol is from about 90 mg/ml to about 98 mg/ml, the ratio of ethanol to water is about 1:5 and the precipitation is carried out at room temperature.

6. A stable lyophilized form of a water insoluble drug suitable for parenteral use prepared by the process as in claim 1.

7. A sterile composition comprising a stable lyophilized form of a water insoluble drug suitable for parenteral use as in claim 1.

8. A kit comprising:
a) a sterile composition as in claim 7, in a first container, and
b) a sterile liquid vehicle consisting essentially of a solubilizer and a solvent selected from organic compounds having a hydroxyl group and molecular weight less than 200, in a second container.

9. A sterile composition prepared by adding a sterile liquid vehicle consisting essentially of a solubilizer and a solvent selected from organic compounds having a hydroxyl group and molecular weight less than 200, to the stable lyophilized form of a water insoluble drug suitable for parenteral use as in claim 6.

10. A sterile composition as in claim 9, wherein the solubilizer is polyoxyethylene 20 sorbitan monooleate and the solvent is ethanol.

11. An infusion solution prepared by a process comprising diluting the sterile composition as in claim 8 with an aqueous infusion vehicle.

12. Stable lyophilized docetaxel.

13. Stable lyophilized docetaxel as in claim 12 having less than 3000 ppm of residual organic solvent.
